(19) United States
(12) Patent Application Publication
(10) Pub. No.: US 2022/0280089 A1
(43) Pub. Date: Sep. 8, 2022

(54) BLADDER OR BOWEL DYSFUNCTION ASSESSMENT SYSTEMS AND METHODS

(71) Applicant: Regents of the University of Minnesota, Minneapolis, MN (US)

(72) Inventors: Dwight E. Nelson, Shoreview, MN (US); Gerald W. Timm, Minneapolis, MN (US); Bhaskar Ravishankar, Minneapolis, MN (US); Ranveer Vasdev, Rochester, MN (US)

(73) Assignee: Regents of the University of Minnesota, Minneapolis, MN (US)

(21) Appl. No.: 17/689,298

(22) Filed: Mar. 8, 2022

Related U.S. Application Data
(60) Provisional application No. 63/158,038, filed on Mar. 8, 2021.

(51) Int. Cl. A61B 5/20; A61B 5/11; A61B 5/00

(52) U.S. Cl. CPC A61B 5/202; A61B 5/1107; A61B 5/4255; A61B 5/4836; A61B 5/7282

(57) ABSTRACT

Systems and methods for the clinical diagnosis of functional bladder states and disorders based upon contractile frequencies and variability. Systems and methods are also provided for the clinical diagnosis of functional bowel states and disorders based upon contractile frequencies and variability.

### RELATED APPLICATIONS

This Non-Provisional patent application claims the benefit of the filing date of U.S. Provisional Patent Application No. 63/158,038, filed Mar. 8, 2021, entitled “BLADDER OR BOWEL DYSFUNCTION ASSESSMENT SYSTEMS AND METHODS,” the entire teachings of which are incorporated herein by reference.

### BACKGROUND

The present disclosure relates to urodynamic (or bowel) testing and assessment. More particularly, it relates to systems and methods for evaluating or quantifying urologic (or bowel) conditions or states of a patient based upon sensed bladder (or bowel) contraction-related information.

The human bladder is a hollow muscular organ connected to the kidneys by ureters. The wall of the bladder is comprised of smooth muscle fibers oriented in multiple directions that collectively define what is known as the detrusor muscle. Contents of the bladder are evacuated through the urethra. The detrusor muscle, and internal and external sphincters control the flow of urine from the bladder through the urethra by what is known as the micturition reflex. In general terms, the detrusor muscle contracts during urination to push urine out of the bladder and into the urethra; the sphincters relax to allow the so-delivered urine to expel from the urethra. The detrusor muscle can then relax to allow the storage of urine in a filling phase. The relaxation to accommodate urine filling occurs during filling and is related to small smooth muscle contraction events that facilitate urine accommodation while monitoring and controlling bladder lumen pressure levels.

Urinary continence entails the ability to store urine in the bladder until the bladder can be appropriately evacuated. Various urologic disorders or conditions may arise. For example, detrusor overactivity (DO) entails involuntary detrusor contractions during the filling phase that may be spontaneous or provoked. DO is divided into idiopathic detrusor overactivity (overactivity when there is no clear cause) and neurogenic detrusor overactivity (overactivity due to a relevant neurological condition). Stress incontinence is another common condition, and includes involuntary leakage of urine from the bladder in response to physical activity (e.g., coughing, laughing, etc.). Other forms of incontinence may be of unknown origin and more generally known as overactive bladder, underactive bladder, or other dysfunctions including mixed in origin and combinations of conditions.

Various urodynamic tests have been developed to assist a treating clinician in better understanding a particular patient's urologic conditions. Understanding the urodynamic performance of a patient's bladder, for example, can be fundamental to effectively diagnose and manage or treat a number of urological disorders and conditions. One conventional urodynamic test utilizes sensors to obtain urodynamic measurements (e.g., pressure readings) while the bladder is artificially filled. For example, a first catheter carrying a pressure sensor is inserted into the patient's bladder and a second catheter also carrying a pressure sensor is inserted into the patient's rectum or vagina. As the bladder is being filled, vesical pressure is measured by the bladder catheter sensor, whereas abdominal pressure is measured by the rectal or vaginal catheter sensor. As a point of reference, the bladder catheter measurement cannot differentiate the pressure generated by the detrusor muscle from that of abdomen contraction; so, to obtain a true identification of detrusor muscle performance, the abdominal pressure reading is subtracted from the vesical pressure reading. Regardless, throughout the test, the patient's subjective reactions as to how his/her bladder feels, whether a need to urinate is occurring, etc., are recorded. From all of this (and perhaps additional) information, the clinician is then tasked with studying all information and making a best guess as the patient's actual condition. These and similar tests may be repeated as the patient is being treated for a determined condition, but do not readily or directly indicate if a treatment regimen is achieving success and/or what changes to the treatment regimen could be beneficial. Similar techniques (e.g., anorectal manometry) are conventionally used to assess possible bowel disorders (e.g., fecal or bowel incontinence).

While well accepted, current clinical urodynamic tests and review too often reply upon patient interviews, questionnaires or diaries, which are not quantitative and are highly susceptible to placebo effect. The conventional techniques are not focused on the pathophysiology of the bladder, but instead on behavior-level measurements such as presence or absence of large detrusor contractions, simple leakage or bladder capacity prior to voiding. Current urodynamic testing as described above can also induce pain and discomfort in the patient as they need to actually result in voiding in order to achieve their measurements of bladder capacity. Further, while inserted sensors may measure bladder pressure or volume, this so-provided sensor information does not quantify disorders, conditions or states using these physiologically relevant signals and signal bands relating to non-voiding contractions.

### SUMMARY

The inventors of the present disclosure recognized that a need exists for improved systems and methods for urodynamic evaluation.

Some aspects of the present disclosure relate to systems and methods for facilitating the clinical diagnosis or evaluation of functional bladder states and disorders based upon contractile frequencies and variability. In some embodiments, the systems and methods of the present disclosure can inform a clinician as to on-going therapy efficacy or modifications, including, for example, long term pharmaceutical treatments or acute device therapies.

### BRIEF DESCRIPTION OF THE DRAWINGS

FIG. 1 is a block diagram of an evaluation system in accordance with principles of the present disclosure;

FIG. 2 illustrates sensors useful with some systems of the present disclosure applied to a patient;

FIG. 3 is a flow diagram of a method in accordance with principles of the present disclosure;

FIG. 4 is a flow diagram of a method in accordance with principles of the present disclosure;

FIGS. 5A-5C diagrammatically illustrate an example of some methods of the present disclosure;

FIG. 6A is a plot of example Spectral Power information generated in accordance with principles of the present disclosure;

FIG. 6B is a plot of example Weighted Average Frequency information generated in accordance with principles of the present disclosure;

FIG. 7A is a plot of example Spectral Power information generated in accordance with principles of the present disclosure, including a representation of a mean Spectral Power at initial and final filling stages of a test session;

FIG. 7B is a plot of example Weighted Average Frequency information generated in accordance with principles of the present disclosure, including a representation of a mean Weighted Average Frequency at initial and final filling stages of a test session;

FIG. 8A is a plot of example Spectral Power information generated in accordance with principles of the present disclosure, including a comparison of Spectral Power at initial and final filling stages of a test session for patients with a diagnosed urological disorder;

FIG. 8B is a plot of example Weighted Average Frequency information generated in accordance with principles of the present disclosure, including a comparison of Weighted Average Frequency at initial and final filling stages of a test session for patients with a diagnosed urological disorder;

FIGS. 9A-9C diagrammatically illustrate an example of some methods of the present disclosure;

FIG. 10A is a plot of example contraction-related data obtained from a patient in accordance with principles of the present disclosure;

FIG. 10B is a CWT scalogram generated from the data of FIG. 10A;

FIG. 11A is a plot of example contraction-related data obtained from a patient in accordance with principles of the present disclosure;

FIG. 11B is a CWT scalogram generated from the data of FIG. 11A;

FIG. 12A is a plot of example contraction-related data obtained from a patient in accordance with principles of the present disclosure;

FIG. 12B is a CWT scalogram generated from the data of FIG. 12A; and

FIGS. 13A-13D are example displays generated by systems and methods of the present disclosure.

### DETAILED DESCRIPTION

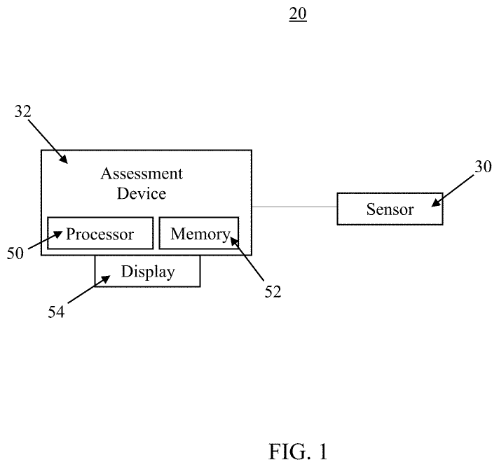

Aspects of the present disclosure relate to bladder or bowel dysfunction evaluation systems and methods. One example of an evaluation system 20 in accordance with principles of the present disclosure, and useful for performing methods of the present disclosure, is shown in block from in FIG. 1. The system 20 includes at least one sensor 30 and an assessment device 32. Details on the various components are provided below. In general terms, the sensor(s) 30 is configured to obtain information from a patient indicative of bladder pressure and/or rectum pressure, and can assume any form conventionally employed. The assessment device 32 is a computer or computer-like device programmed to derive tissue contraction information from the sensor data during a test session in which the organ of interest (e.g., bladder or rectum) is at least partially filled over time, and establish a quantitative diagnostic evaluation of functional and dysfunctional states or conditions of the bladder or bowel based upon physiology and pathophysiology.

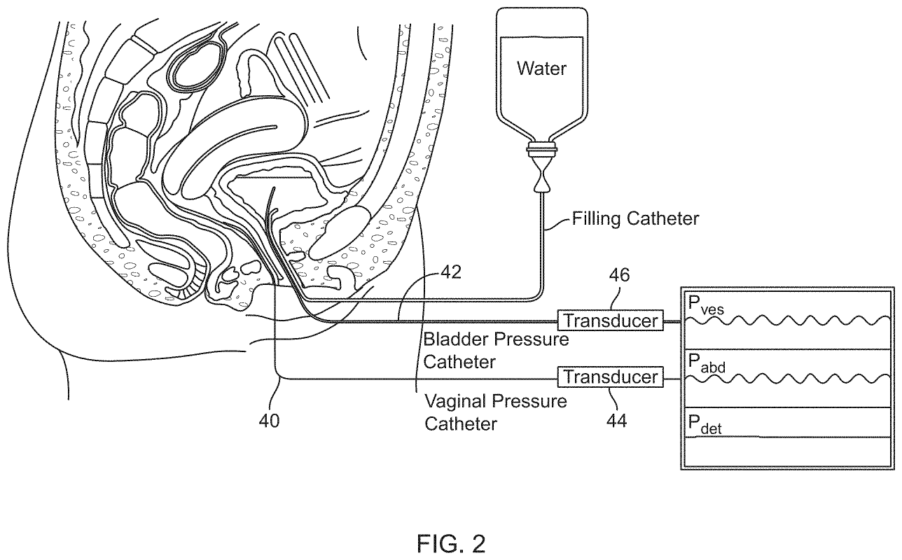

The sensor(s) 30 can include, in some embodiments, catheter-based sensors conventionally used with urodynamic testing. For example, the sensor 30 can be a pressure-sensing air balloon (or other sensor format), transducer, etc., carried by a catheter sized and shaped for insertion into a patient's bladder, rectum, vagina, etc. Consistent with these explanations, then, the sensor(s) 30 can be connected to one or more other components typically used to locate and operate the sensor 30 as desired. One non-limiting example of a sensor arrangement useful with the present disclosure is shown in FIG. 2 as applied to a patient, and includes a bladder pressure catheter 40 and a vaginal pressure catheter 42. When installed to the patient, sensors 44, 46 provided with the catheters 40, 42 generate signals indicative of vesical pressure and abdominal pressure, respectively. Returning to FIG. 1, other pressure or contraction sensing devices are also envisioned. For example, the sensor(s) 30 can be provided as part of a currently-available implanted or temporary bladder sensor device, configured to sense and generate signals from which bladder pressure and/or contraction can be derived, such as EMG, peri-bladder sensing, accelerometer, visible motion, auditory sensor or microphone, etc.

The assessment device 32 is configured to produce quantitative evaluation data or information associated with a patient being tested in accordance with the methods or protocols described herein based upon information from the sensor(s) 30. The assessment device 32 includes a processor 50 coupled to one or more memories 52. The processor 50 can be a microprocessor, and embedded microprocessor, and embedded controller, a digital signal processor (DSP), etc. The processor 50 is configured to execute program code stored as software in the memory 52. The program code, when executed by the processor 50, causes the processor 50 to implement the assessment functions described herein. The processor 50 can further cooperate with the memory 52 to store data received from the sensor(s) 30. In this regard, the assessment device 32 can include one or more features for receiving data from the sensor(s) 30 either directly (e.g., wired or wireless interface with the sensor(s) 30) or indirectly (e.g., sensor information collected during a test session can be separately stored and then loaded to the assessment device 32). The assessment device 32 can optionally include, or be connected to, additional components for conveying information to and/or receiving information from a user, such as a display 54.

In some non-limiting examples, the systems and methods of the present disclosure entail a test session akin to urodynamic cystometric testing in which vesical and abdominal pressures are sensed while the patient's bladder is incrementally filled (though not necessarily completely filled to the point of voiding). In this regard, it has been surmised that during bladder filling, there are detectable differences in the frequencies of bladder wall (or detrusor) contractions that may reflect rhythmic or similar dynamic changes in the bladder walls. The spontaneous contractions appear to be associated with small amplitude pressure changes otherwise known as low amplitude rhythmic contractions or non-voiding contractions (small bladder contraction pressure waves) in the pressure range of <15 cm H₂O. These non-voiding contractions can be linked to pressure regulation during storage/filling, and dysfunctions such as detrusor overactivity, retention, and neurogenic bladder. Additional contraction events and frequencies may be involved with other functions (and/or dysfunctions) of the bladder While some efforts have been made to quantify non-voiding contraction signals and other dynamic signals in the bladder, currently there is no scalable or quantifiable parameter available to treating clinicians or for scientific use. With this in mind, some embodiments of the present disclosure include analyzing the non-voiding contraction signals obtained during the testing period to provide a quantitative measure of bladder state or bowel state.

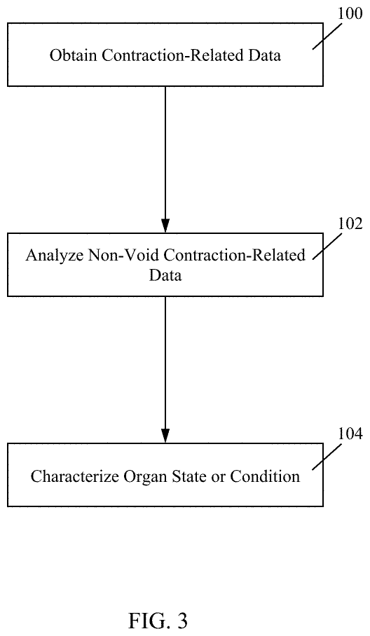

With this in mind, and with reference to FIG. 3, some methods of the present disclosure can include, at step 100, obtaining bladder or bowel contraction-related sensor data, for example over the course of a filling test session (e.g., cystometry). In some non-limiting examples described above, the contraction sensor data can include or comprise bladder or bowel pressure-related sensor data, for example vesical and abdominal pressure data, although other contraction-related sensor information is also acceptable. Regardless, the contraction-related sensor data from the test prior to the patient voiding is identified and analyzed at step 102. As a point of reference, the filling test session may or may not including filling the bladder or rectum to the point of voiding; under circumstances where voiding occurs as part of the filling test session, only contraction-related sensor data from points in time prior to voiding considered. Various analyses of the present disclosure are described in greater detail below. In general terms, the non-voiding contraction data is transformed to provide a quantitative measure of bladder state or bowel state. In other embodiments, the methods of the present disclosure can include, at step 100, obtaining bladder or bowel contraction-related sensor data from a patient in situ, without necessarily subjecting the patient to an active filling (or cystometry) test. Thus, while several non-limiting examples are described below in the context of a filling test, in other embodiments, the systems and methods of present disclosure do not entail a filling test, but rather include monitoring a patient's bladder or bowel over time as described below (e.g., for use with diagnostics, closed loop therapy, etc.). At step 104, the quantitative measure of bladder state or bowel state is reviewed to characterize one or more of a current clinical condition of the patient, an effectiveness of current therapy being provided to the patient, recommended change to a current therapy being provided to the patient (e.g., therapy titration and dosing), etc.

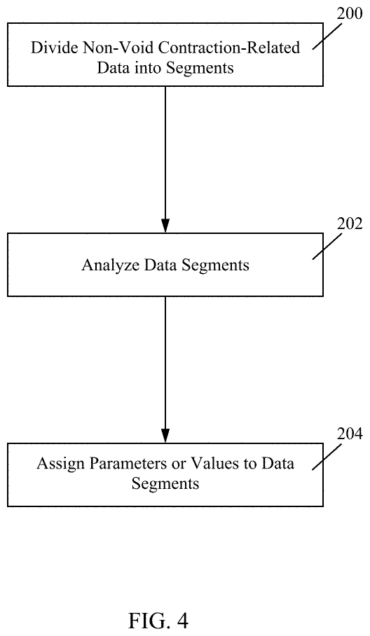

The analysis performed at step 102 can take various forms. In some embodiments, a mean spectral power during bladder (or bowel) filling and a mean weighted average frequency are derived from the bladder (or bowel) pressure-related sensor information as a quantitative measure of bladder (or bowel) state. The inventors of the present disclosure have surprisingly found that with a normal bladder, the mean spectral power for bladder pressure dynamics will change during bladder filling, as will the mean average frequency. With this in mind, and with reference to FIG. 4, the analysis can include dividing the non-void contraction-related data into equal bins or segments at step 200. In some embodiments, the non-void data can be divided into 300 second bins/segments, although other ranges are acceptable. The data segments are then analyzed at step 202. In some embodiments, the analysis at step 202 includes calculating a Fast Fourier Transform (FFT) of the data segment. With optional embodiments in which the sensor data includes vesical and abdominal pressure signals, the segment analysis at step 202 can further include a spectral subtraction in which the abdominal FFT is subtracted from the vesical FFT to obtain a bladder FFT. Regardless, at step 204, the power output of the transformed oscillatory signals is summed over a designated frequency range, for example 1-10 cycles per minute (CPM), and is assigned summary values or parameters such as Spectral Power (summed over the designated CPM range) and Weighted Average Frequency (a weighted average frequency detected over the designated CPM range). Equation 1 below provides Spectral Power for a 1-10 CPM, and Equation 2 provides Weighted Average Frequency for a 1-10 CPM.

$$Spectral Power = {\sum }_{f = 1}^{10} P ( f ) \qquad Eq . 1$$

$$Wt . Avg . Freq = \frac{{\sum }_{f = 1}^{10} \{ P ( f ) \times f \}}{{\sum }_{f = 1}^{10} P ( f )} \qquad Eq . 2$$

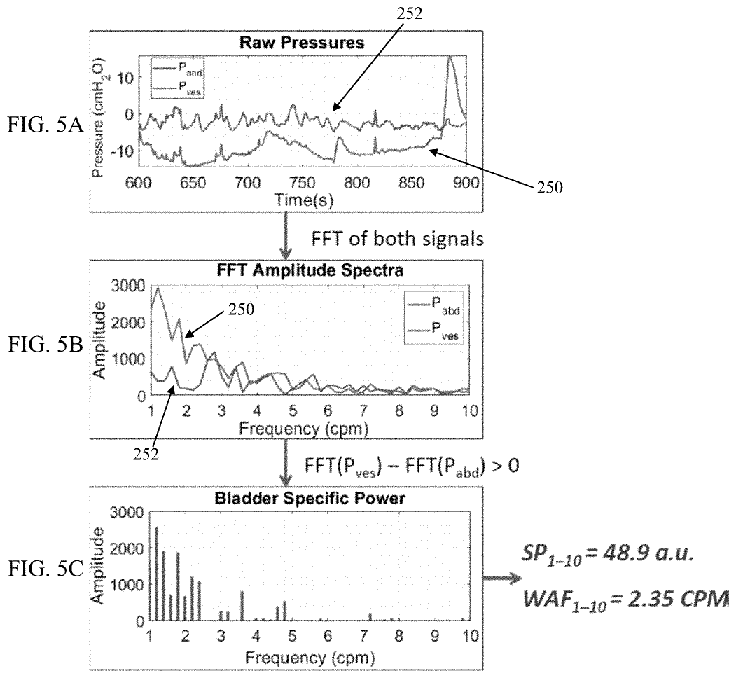

The so-determined Spectral Power and Weighted Average Frequency values provide a quantified representation of the patient's bladder (or bowel) state or functioning. The determined Spectral Power and/or Weighted Average Frequency can be compared with standardized values to implicate a particular bladder (or bowel) dysfunction. By way of further explanation, FIG. 5A is an example of pressure data (vesical and abdominal pressures) obtained from a human patient over the course of a bladder filling test session (vesical pressure is plot line 250; abdominal pressure is plot line 252). FIG. 5B reflects calculated FFT for the two pressure readings of FIG. 5A for frequency range of 1-10 cycles per minute. FIG. 5C graphically reflects subtraction of the abdominal FFT from the vesical FFT of FIG. 5B, and from which Spectral Power and Weighted Average Frequency can be calculated via Equations 1 and 2 above.

In some embodiments, the Spectral Power and Weighted Average Frequency can be considered across various stages or segments of the filling test session. In this regard, the inventors of the present disclosure have surprisingly found that with a healthy or normal bladder, the Spectral Power increases significantly with bladder filling, and the Weighted Average Frequency decreases significantly with bladder filling. Further, both Spectral Power and Weighted Average Frequency show statistically significant differences between initial and final fill segments in a healthy or normal bladder. Thus, where the Spectral Power and/or Weighted Average Frequency for a particular patient do not exhibit all of these attributes, it is readily apparent that the patient is suffering from a bladder or other urologic disorder. Further, ratios of lower frequency and higher frequency can be defined or implemented that may be useful for comparing bladder filling level in normal patients (e.g., without detrusor overactivity). A similar comparison can be made for review of bowel state or functioning. For example, a wide range of frequencies on the order of 0.1-20 cycles per minute (CPM) can be available for consideration in some embodiments. From this, comparisons can be made as 0.1-6 CPM versus 6-20 CPM; alternatively as 0.1-10 CPM versus 10-20 CPM; alternatively as 1-5 CPM versus 5-10 CPM; alternatively as 1-3 CPM versus 3-6 CPM; etc. Comparisons within the designated ranges can be for one or more different parameters. For example, power, other forms or formats of spectral analyses, shapes of spectra, etc.

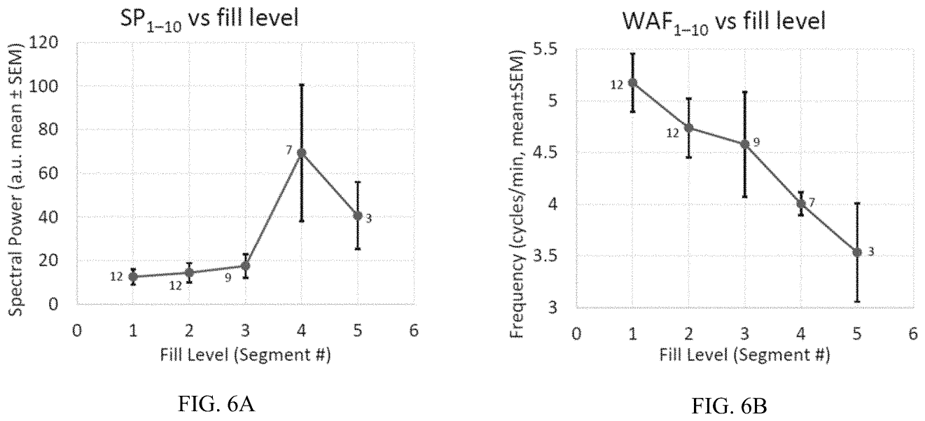

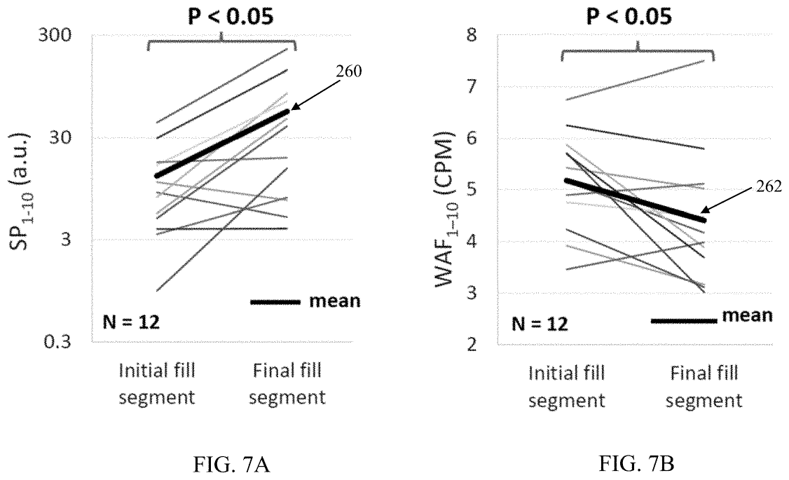

By way of further explanation, FIG. 6A is an example of determined Spectral Power values vs. fill level segment obtained for twelve patients using the testing protocol described above; FIG. 6B provides the determined Weighted Average Frequency values vs. fill level segment obtained for these same twelve patients. From the information of FIG. 6A, FIG. 7A graphically reflects the change in determined Spectral Power from the initial fill segment to the final fill segment, and highlights the mean Spectral Power (plot line 260). From the information of FIG. 6B, FIG. 7B graphically reflects the change in determined Weighted Average Frequency from the initial fill segment to the final fill segment, and highlights the mean Weighted Average Frequency (plot line 262). As shown, the mean Spectral Power increased substantively from the initial fill segment to the final fill segment, whereas the Weighted Average Frequency experienced a substantive decrease.

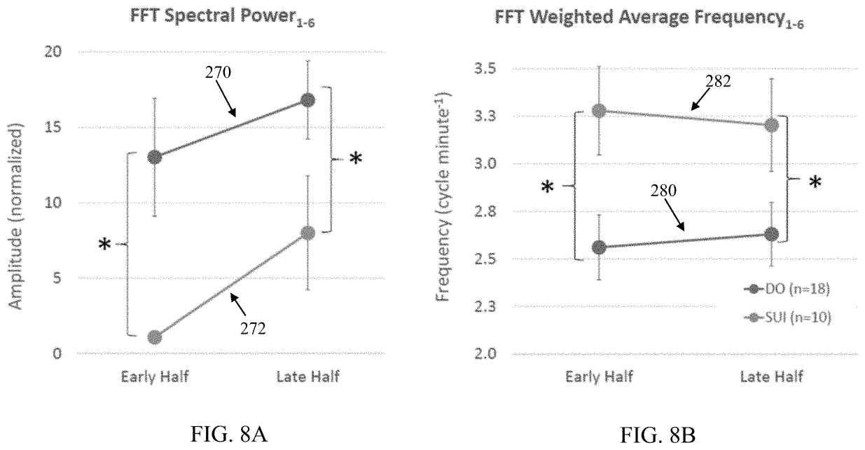

Further confirmation of the Spectral Power and/or Weighted Average Frequency analyses of the present disclosure providing a quantitative identification of a urological dysfunction was provided by analyzing vesical pressure and abdominal pressure values resulting from a filling test applied to a grouping of patients. Eighteen of the patients of this study were known to suffer from detrusor overactivity (“DO”), and ten of the patients of this study were known to suffer from stress urinary incontinence (“SUI”) and thus are correctly classified as “non-DO” patients. The patient test sessions were reviewed at a range of 1-6 cycles per minute, and the resultant pressure signals were divided into early and late segments of filling. The Spectral Power and Weighted Average Frequency values were then determined for both of the early and late segments of filling for each of the patients of the study. FIG. 8A is a comparison of the mean Spectral Power of the eighteen DO patients at the early half and late half of filling (plot line 270) with the mean Spectral Power of the ten non-DO patients at the early half and late half of filling (plot line 272). FIG. 8B is a comparison of the mean Weighted Average Frequency of the eighteen DO patients at the early half and late half of filling (plot line 280) with the mean Weighted Average Frequency of the ten non-DO patients at the early half and late half of filling (282). As reflected by FIGS. 8A and 8B, the Spectral Power and Weighted Average Frequency surprisingly differed significantly for DO patients and compared to non-DO patients. From these and other reviews, the Spectral Power and/or Weighted Average Frequency analyses of the present disclosure can be employed to readily diagnose a urological disorder, such as DO or non-DO, by comparing the Spectral Power and/or Weighted Average Frequency obtained for a particular patient under review with a predetermined template or model, such as that of FIG. 8A and/or FIG. 8B.

Alternatively or in addition, the filling test as described above can be used with a patient receiving therapy or treatment for urologic disorder at various points over the course of the treatment program; a comparison of the determined Spectral Power and/or Weighted Average Frequency from a later test session with an earlier test session can readily apprise the clinician as to the success of the treatment program. Further, depending upon the change (or lack thereof) in Spectral Power and/or Weighted Average Frequency over time, the treating clinician can determine possible changes to the therapy program.

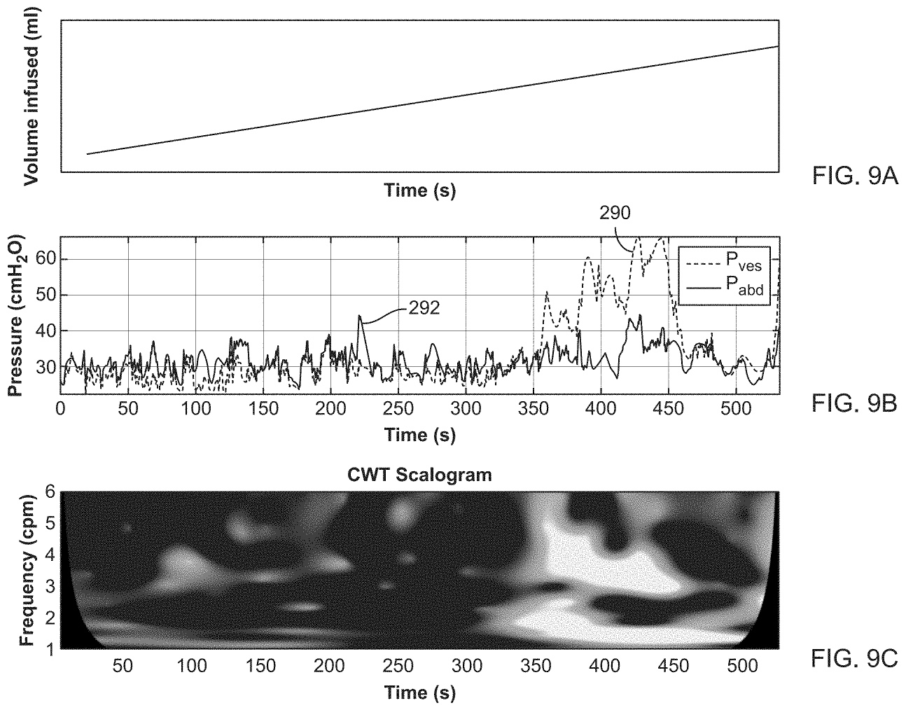

In other embodiments, the analysis performed at step 102 (FIG. 3) can include a wavelet transform of the contraction-related data alone, or in combination with the FFT analysis described above. The so-obtained wavelet transform (e.g., continuous wavelet transform (CWT) scalogram plot) can then be compared against wavelet transform information associate with a health or normal bladder to ascertain likelihood of dysfunction. By way of further explanation, FIGS. 9A, 9B, and 9C are examples of information generated or obtained during a filling test session as described above performed on human patient. FIG. 9A is the filling volume over time; FIG. 9B plots the obtained vesical pressure readings (plot line 290) and abdominal pressure readings (plot line 292); FIG. 9C is the corresponding, determined CWT scalogram. The wavelet analysis, the Spectral Power, and/or the Weighted Average Frequency can directly implicate a particular urologic disorder.

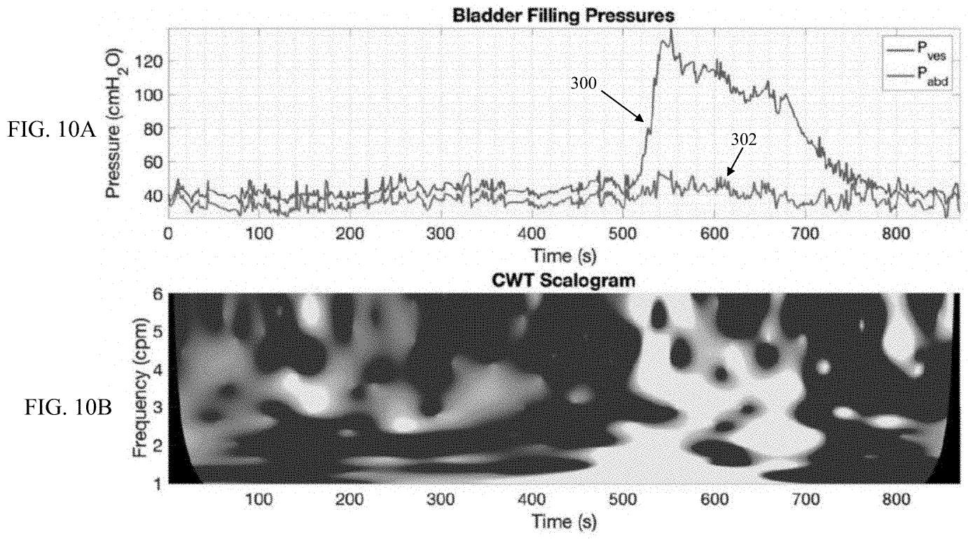

For example, the patient associate with the information of FIGS. 9B and 9C was known to suffer from detrusor overactivity. Another patient also suffering from detrusor overactivity was subjected to the same testing protocols, with the test session resulting in the pressure readings and CWT scalogram of FIGS. 10A and 10B, respectively. In FIG. 10A, the vesical pressure readings are represented by plot line 300; the abdominal pressure readings are represented by plot line 302. At least the CWT scalograms (FIGS. 9C and 10B) exhibit similar characteristics that can be utilized when considering the CWT scalogram (or other wavelet analysis) of a patient requesting an initial diagnosis for possible detrusor overactivity. Alternatively or in addition, the patient associated with the information of FIGS. 9B and 9C (or of FIGS. 10A and 10B) otherwise being treated for detrusor overactivity can be subjected to the same test at a later point in time; a comparison of the generated CWT scalograms can provide a clear indication of therapy success (or lack thereof).

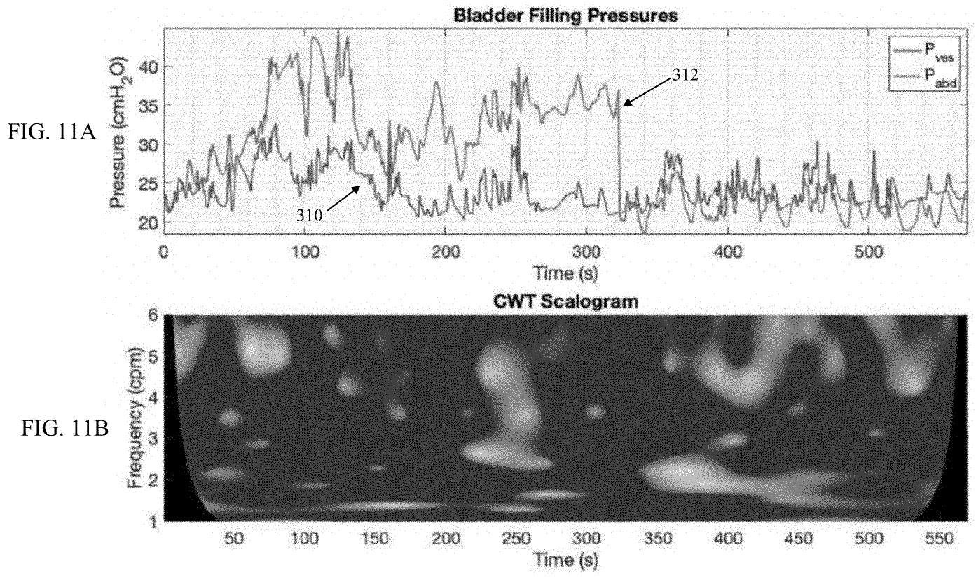

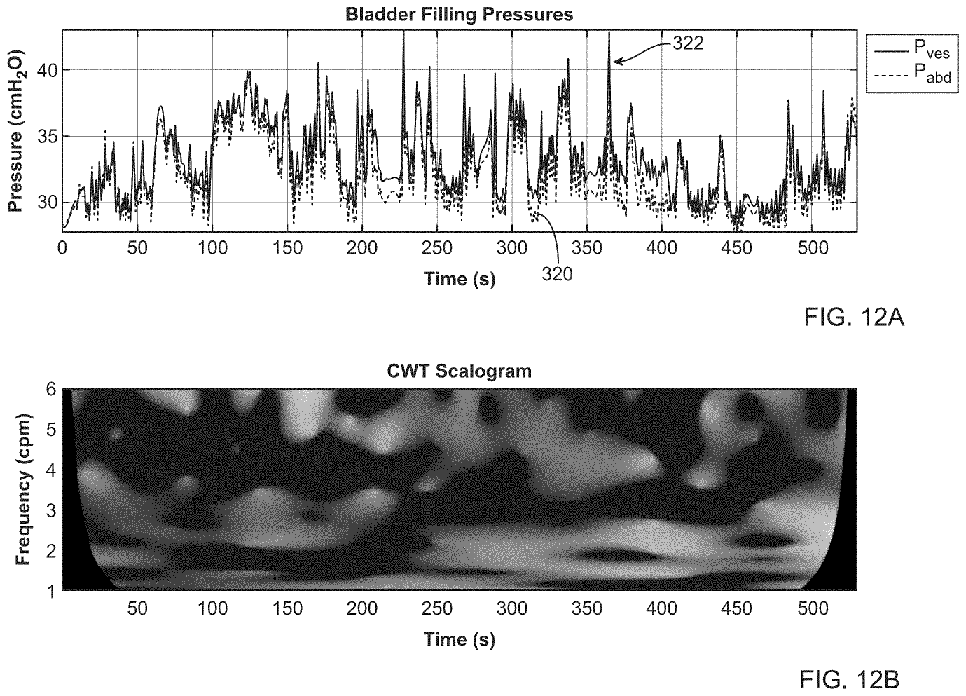

As a further explanation, FIGS. 11A and 11B illustrate the pressure readings and CWT scalogram obtained using the testing protocols above for a first patient previously confirmed as not suffering from detrusor overactivity. In FIG. 11A, the vesical pressure readings are represented by plot line 310; the abdominal pressure readings are represented by plot line 312. FIGS. 12A and 12B illustrate the pressure reading and CWT scalogram obtained using the same testing protocols for a second patient who also was previously confirmed as not suffering from detrusor overactivity. In FIG. 12A, the vesical pressure readings are represented by plot line 320; the abdominal pressure readings are represented by plot line 322. At least the CWT scalograms of the “non-detrusor overactivity” patients (i.e., FIGS. 11B and 12B) were surprisingly found to be markedly different from the CWT scalograms of patients known to be suffering from detrusor overactivity (i.e., FIGS. 9C and 10B), thus confirming that the wavelet transform analysis techniques of the present invention can useful to readily implicate or diagnose detrusor overactivity.

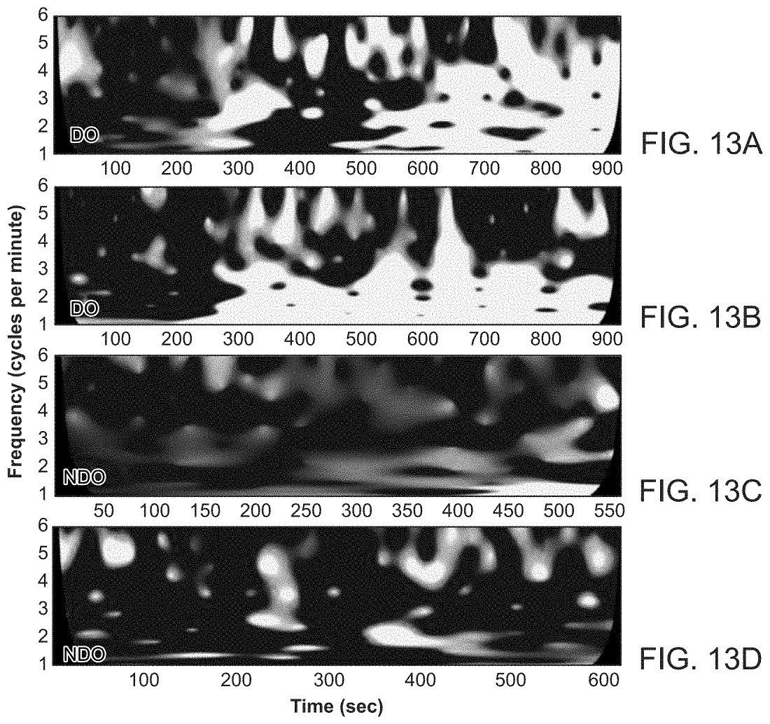

Returning to FIG. 1, following performance of a patient test session, the assessment device 32 can be programmed to present or display the contraction-related analyses in various forms. For example, in some embodiments, the assessment device 32 is programmed to generate one or more of the analyses (e.g., Spectral Power as a single quantitative number, Spectral Power as segmented over the test session, Weighted Average Frequency as a single quantitative number, Weighted Average Frequency as segmented over the test session, a wavelet analysis (e.g., CWT scalogram), etc.) in a form appropriate for review by a clinician (e.g., at the display 54) who then evaluates the so-provided results (e.g., diagnosis the patient, evaluates an effectiveness of a therapy regimen being delivered to the patient, determines changes to a therapy regimen being delivered to the patient, etc.). By way of non-limiting example, the assessment device 32 can be programmed to show or display (e.g., at the display 54) one or more images or graphics of the collected signals information to a clinician for use in diagnosis (e.g., the assessment device 32 may not, in some embodiments, quantify or predict a condition of the patient, but more simply graphically present the data to allow a clinical decision). Non-limiting examples of possible displays or graphics that could be displayed to the clinician are provided in FIGS. 13A-13D. As a point of reference, each of the displays or graphics of FIGS. 13A-13D present collected data at a frequencies of 1-6 CPM (y axis) over time or fill level (x axis). As a point of reference, a clinician reviewing or interpreting the display or graphic of FIG. 13A or FIG. 13B could conclude that the patient in question suffers from DO; a clinician reviewing or interpreting the display or graphic of FIG. 13C or FIG. 13D could conclude that the patient in question does not suffer from DO. In other embodiments, the assessment device 32 can be programmed to compare one or more of the analyses (e.g., Spectral Power as segmented over the test session, a Weighted Average Frequency as segmented over the test session, a wavelet analysis (e.g., CWT scalogram)) with one or more predetermined templates or models stored in the memory 52 to estimate a state or possible urologic (or bowel) dysfunction of the patient, and report such estimations to the clinician, for example at the display 54. In other embodiments, the assessment device 32 can be programmed to compare one or more of the analyses (e.g., Spectral Power as segmented over the test session, a Weighted Average Frequency as segmented over the test session, a wavelet analysis (e.g., CWT scalogram)) with previous test session results for the patient stored in the memory 52 to determine an effectiveness of a therapy regimen being delivered to the patient, determine changes to a therapy regimen being delivered to the patient, etc., and report such estimations to the clinician, for example at the display 54.

The analyses described above are non-limiting examples of evaluation techniques of the present disclosure. In other embodiments, for example, broader uses and spectral characterizations are also possible by comparing specific frequency ranges, etc. In other embodiments, the analysis performed at step 102 (FIG. 3) or step 202 (FIG. 4) can include alternative or additional filtering of collected contraction signal data (e.g., an alternative to (or in addition to) subtracting abdominal pressure signal or data from vesical pressure signal or data to obtain a bladder (or bowel) signal or data). For example, in some embodiments, signal or data from an accelerometer or the like (e.g., other “noise” frequency spectra signals such as pressure sensor, gyroscope sensor, stretch mechanical sensor, audio sensor, etc.) installed to the patient is subtracted from the bladder or bowel signal to capture bladder-specific or bowel-specific frequency spectra and signals. It has surprisingly been found that this so-derived signal (e.g., vesical pressure signal minus noise frequency spectra signal) can provide a more meaningful evaluation of, for example, possible DO.

Some systems and methods of the present disclosure can optional incorporate one or more of an accelerometer, gyroscope or similar motion sensing capabilities that will allow rapid and automated annotation. As a point of reference, motion or mechanical artifacts may disrupt or overwhelm the signals described and in clinical measurements these movements can be significantly large to obscure interpretation using algorithms or even visual interpretation. Use of movement sensing can be used to flag, mark or similarly annotate the data signals to allow automated or manual exclusions or similar correction methods to be used to clean or de-noise the data. These signals, as well as the rapid or real-time analyses of the pressure spectra, can provide feedback to the technician or an automated data capture system that can ensure adequate analyzable durations of data can be captured during a measurement session in some embodiments.

As mentioned above, some systems and methods of the present disclosure can include devices and/or steps akin to typical urodynamics filling test. In this regard, pump control in current urodynamics is linear or similarly constant, such as 20 ml per minute or 10 ml per minute. In some embodiments, improved methods of pump control will allow the analytics system to rapidly change the filling rate including inflow and outflow patterns, zero fill durations with no filling or removal of saline, or other patterns of filling including ramped filling, sinewave oscillations in filling/removal or filling and reduced filling, and similar patterns of pressure, infusion/effusion, or pump control based upon detected dynamics signals or other methods.

In some optional embodiments, the systems and methods of the present disclosure can include the use or implementation of artificial intelligence (“AI”) or machine learning. Incorporation of AI or machine learning (“ML”) approaches into the analyses of the spectral dynamics can allow algorithm training to detect novel signals and methods to determine bladder detrusor activity, underactive bladder or other conditions relating to bladder filling or voiding. These computational techniques could be used in parallel with other features of the present disclosure for diagnostics, measurement of disease severity, assessment of therapeutic efficacy, etc. For example, AI or ML could be used to in parallel leverage FFT derived markers (e.g., markers related to bladder contraction signal frequencies).

The bladder or bowel dysfunction evaluation systems and methods of the present disclosure provide a marked improvement over previous designs. With the systems and methods of the present disclosure, functional and dysfunctional states or conditions of a patient's bladder or bowel can be quantified based upon physiology and pathophysiology. Unlike existing urological testing protocols, the systems and methods of the present disclosure quantify disorders, conditions or states using physiologically relevant signals and signal bands related to non-voiding contractions (fairly low frequency, oscillatory signals). Further, and unlike existing urological testing protocols, the systems and methods of the present disclosure provide meaningful urological (or bowel) evaluations using non-filled bladders (e.g., not required to fill the bladder to the point of voiding), reducing patient discomfort, such as urinary urge, during testing.

In addition to human and clinical use, the pressure spectra and dynamics of the present disclosure can be utilized in preclinical testing of products, therapies, treatments and other uses related to producing clinically useful human treatments. Preclinical use of the procedure and processes can be used in large animal (e.g. sheep, pig, cattle, or similar animal models of similar size scale to humans) and can include screening of therapies, optimization of therapies and to improve therapy efficacy more broadly. Outcomes from this work at the preclinical level will allow improved and increased success of early clinical testing by providing appropriate and relevant design inputs for human therapies, preliminary data and experience and similar outcomes.

Use of the pressure spectral dynamics of the present disclosure can also be applied, in some embodiments, to testing of therapies to reduce DO or otherwise treat this disorder. For example, pressure spectra can be obtained from patients or animals during bladder filling while also applying an acute therapy or treatment (or prior/following treatment or therapy) and responses or changes of bladder pressure spectra can be used to determine the relative effectiveness of the therapy or treatment, improve the therapy or treatment through targeting or dosage of the treatment or for patient specific titration or testing of the treatment or therapy. Possible therapies or treatments include nerve or tissue stimulation using electrical fields or current applications to tissue or body surface, stem cell or gene therapy applications, ablation of tissue, pharmacologic or biologic treatments or other methods. The systems and methods of the present disclosure can be applied, in some embodiments, with chronic sensing of bladder spectral dynamics for therapy monitoring, titration, closed loop, etc. For example, rather than (or in addition to) generating the analyses described above as part of a filling test, the systems and methods of the present disclosure can be utilized on an on-going basis for a patient, for example to assist in selecting therapeutic parameters, dosages, or delivery of therapies or treatments and effective timing of those treatments to an animal or human patient.

Although the present disclosure has been described with reference to preferred embodiments, workers skilled in the art will recognize that changes can be made in form and detail without departing from the spirit and scope of the present disclosure.

What is claimed is:

1. A method for evaluating a state of at least one of a patient's bladder and a patient's bowel, the method comprising:
receiving data indicative of contraction at least one of a patient's bladder and a patient's bowel over the course of a testing session in which at least one of the bladder and the bowel is monitored;
analyzing low frequency, oscillatory signals in the received data; and
characterizing the at least one of the bladder and the bowel based upon the analysis.

2. The method of claim 1, wherein the step of analyzing includes determining Spectral Power.

3. The method of claim 1, wherein the step of analyzing includes determining Weighted Average Frequency.

4. The method of claim 1, wherein the step of analyzing includes generating a CWT scalogram.

5. The method of claim 1, wherein the step of characterizing includes comparing the analyses with a predetermined model.

6. The method of claim 1, wherein the step of receiving data is performed while subjecting the patient to a filling test.

7. The method of claim 1, further comprising selecting at least one of therapeutic parameters, dosages, delivery of therapies, treatment, and effective timing of treatment to the patient based upon the characterization.

8. The method of claim 7, wherein the step of selecting is performed on a closed loop basis.

9. The method of claim 1, wherein the step of receiving includes receiving data indicative of contraction of the patient's bladder, and further wherein the step of characterizing includes characterizing the patient's bladder based upon the analysis.

10. The method of claim 1, wherein the step of receiving includes receiving data indicative of contraction of the patient's bowel, and further wherein the step of characterizing includes characterizing the patient's bowel based upon the analysis

11. A method for evaluating a state of at least one of a patient's bladder and a patient's bowel, the method comprising:
receiving data indicative of contraction of at least one of a patient's bladder and a patient's bowel over the course of a testing session in which at least one of the bladder and the bowel is monitored;
analyzing low frequency, oscillatory signals in the received data; and
applying the analysis to at least one of titrating, targeting, screening, optimizing and monitoring of therapeutic effects of a treatment in the patient.
